(19) United States
(12) Patent Application Publication
(10) Pub. No.: US 2004/0082013 A1
(43) Pub. Date: Apr. 29, 2004

(54) Methods for screening for substances which inhibit fp prostanoid receptor interaction with a compound having pgf2alpha activity and methods of treating cancer

(76) Inventor: John W Regan, Tuscon, AZ (US)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/466,545

(22) PCT Filed: Jan. 24, 2002
(86) PCT No.: PCT/US02/00522

(51) Int. Cl. G01N 33/53; G01N 33/567; C12Q 1/68

(52) U.S. Cl. 435/7.2; 435/6

(57) ABSTRACT

The present invention provides mehtods for screening for substances which inhibit the interaction between a FP prostanoid receptor and a compoudn having PGF_{2αalpha}activity, methods for inhibiting the interaction, methods of inhibiting signaling mediated by βbeta-catenin, and methods of treating cancer.

### BACKGROUND OF THE INVENTION

1. Field of the Invention

The present invention provides methods for screening for substances which inhibit the interaction between FP prostanoid receptors and a compound having PGF_{2α}activity, methods for inhibiting the interaction, methods of inhibiting signaling mediated by β-catenin, and methods of treating cancer.

2. Discussion of the Background

The primary amino acid sequences of the ovine FP_{A} and FP_{B} prostanoid receptor isoforms are the same throughout their amino termini and seven membrane spanning domains, but the FP_{B} isoform is truncated and lacks the last 46 carboxyl terminal amino acids present in the FP_{A} isoform (1). This is very similar to the EP₃ (2) and thromboxane A2 (3) prostanoid receptors in which alternative mRNA splicing gives rise to a variety of isoforms in humans and in other species (4). The physiological significance of these receptor isoforms is not clear, although differences have been shown to exist with respect to second messenger coupling and receptor desensitization. The inventor has discovered that the FP_{A} and FP_{B} receptor isoforms have similar pharmacological properties and that prostaglandin F_{2α} (PGF_{2α})¹ stimulates phosphoinositide turnover to a similar extent in cells expressing these isoforms (1). In addition, stimulation of FP_{A} or FP_{B} expressing cells with PGF_{2α} activates Rho leading to the formation of actin stress fibers, phosphorylation of p125 focal adhesion kinase and cell rounding (5). Cell rounding involves the retraction of filopodia and a change from an isolated dendritic appearance to one in which the cells are rounded and form small cobblestone-like aggregates (see FIG. 1A). Following the removal of PGF_{2α}, however, FP_{A} expressing cells return to their original dendritic morphology, but the FP_{B} expressing cells do not and remain rounded (6). Here we show that Tcf/β-catenin mediated transcriptional activation is elevated 16 hours after an initial 1 hour treatment of FP_{B} expressing cells with PGF_{2α}. This is not observed in FP_{A} expressing cells and suggests that FP_{B} expressing cells remain rounded because of persistent activation of a Tcf/β-catenin signaling pathway.

Previous studies by others have established that stimulation of this pathway is strongly associated with the development of colorectal and other cancers. It is also well known that the inhibition of cyclooxygenase enzymes by nonsteroidal anti-inflammatory drugs (NSAIDs) is beneficial for the treatment and prevention of colorectal cancer. This beneficial effect is assumed to result from the decreased biosynthesis of prostanoid metabolites, however, since dozens of metabolites are affected, the specific mechanism whereby this decrease produces a benefit is unknown. Our data are the first to provide an unambiguous receptor-based mechanism whereby a decrease in a specific prostanoid metabolite, PGF_{2α}, could account for the beneficial effects of NSAIDs in the prevention and treatment of colorectal cancer. In terms of this receptor-based mechanism, it can be predicted with virtual certainty that a FP prostanoid receptor antagonist would have the same functional consequence as selectively decreasing PGF_{2α} Thus, there is a reasonable expectation that FP prostanoid receptor antagonists would be effective as drugs in the treatment and prevention of colorectal cancer and possibly other cancers as well. It is also clearly evident that the use of recombinant FP prostanoid receptors in functional screens would be an effective means of discovering existing and novel substances that could be used as such drugs. The present technology based on the use of NSAIDs is nonspecific because NSAIDs block the key enzymes (cyclooxygenases) required for the biosynthesis of all prostanoid metabolites. Because so many metabolites are affected, it is actually very uncertain as to how the NSAIDs are producing their beneficial actions. In addition, the use of NSAIDs is associated with a number of adverse effects related to their widespread effects on prostanoid metabolite biosynthesis. Presently, as it concerns the treatment and prevention of cancer, there is no existing technology based on the pharmacological blockade of a specific prostanoid receptor subtype or isoform. Furthermore, there are no existing data that we are aware of that would even support such an approach if it were contemplated. Our disclosure is novel because it clearly establishes the feasibility of using prostanoid receptor antagonists for the treatment and prevention of cancer. It would be expected that such prostanoid receptor antagonists would have the potential to be more efficacious with fewer adverse side effects. In addition, the use of recombinant FP receptors for the discovery of potential anti-cancer drugs is unprecedented because until now there was no obvious reason to expect that FP receptors might be involved with the pathophysiology of cancer. The use of recombinant FP receptors for such a purpose would have the significant advantages because the present technology for the discovery of potential anticolorectal cancer drugs are highly nonspecific and do not take into account this receptor-based mechanism for the treatment of this disease.

### SUMMARY OF THE INVENTION

One object of the present invention is a method of screening for substances which inhibit the interaction between a FP prostanoid receptors and a compound having PGF_{2α} activity including contacting a cell expressing the FP prostanoid receptors with the substance to be screened, contacting the cell with said PGF_{2α} compound, and assaying the presence or absence of interaction between FP prostanoid receptors and said PGF_{2α} compound, wherein the absence of an interaction between FP prostanoid receptors and said PGF_{2α} compound indicates the substance inhibits the interaction.

In a preferred embodiment the FP prostanoid receptor is FP_{B.} and said PGF_{2α} compound is PGF_{2α}.

Another object of the present invention is assaying with a detection method selected from RT-PCR, Northern blot, luciferase reporter gene, β-gal reporter gene, and other reporters. Another object of the present invention is where the inhibiting substance is an antibody. Such an antibody can bind to FP prostanoid receptors or said PGF_{2α} compound.

Another object of the present invention is a method of screening for substances which inhibit the interaction between a FP prostanoid receptors and a compound having PGF_{2α} activity by introducing and expressing a polynucleotide which encodes the FP prostanoid receptors; contacting the cell expressing the FP prostanoid receptors with the substance to be screened; contacting said cell with said PGF_{2α} compound; and assaying the presence or absence of interaction between FP prostanoid receptors and said PGF_{2α} compound, wherein the absence of an interaction between FP prostanoid receptors and said PGF_{2α} compound indicates the substance inhibits the interaction.

Another object of the present invention is a method of inhibiting the interaction between FP prostanoid receptors and a compound having PGF_{2α} activity ound by contacting said FP prostanoid receptors with a substance which is capable of inhibiting the interaction.

Another object of the present invention is a method of inhibiting β-catenin signaling by contacting a cell expressing FP prostanoid receptors with a substance which is capable of inhibiting the interaction between FP prostanoid receptors and a compound having PGF_{2α} activity.

Another object of the present invention is a method of inhibiting G12 and G13 mediated signaling by contacting a cell expressing FP prostanoid receptors with a substance which is capable of inhibiting the interaction between FP prostanoid receptors and a compound having PGF_{2α} activity.

Another object of the present invention is a method of treating cancer by administering to a patient a substance which inhibits the interaction between FP prostanoid receptors and a compound having PGF_{2α} activity in an amount sufficient to inhibit the interaction.

Another object of the present invention is a method of screening for substances which inhibit β-catenin signaling by contacting a cell expressing FP prostanoid receptors with the substance to be screened; contacting said cell with a compound having PGF_{2α} activity; assaying the signaling activity, phosphorylation and/or the subcellular localization of the β-catenin; wherein a change in one or more of these properties indicates the substance inhibits the interaction β-catenin signaling.

Another object of the present invention is method of screening for a substance for their ability to inhibit cancer cell growth by contacting a cell expressing FP prostanoid receptors with the substance to be screened; contacting the cell with PGF_{2α}; assaying the change in cell growth, wherein a decrease in cell growth is indicative of the substance usefulness for the treatment of cancer. In one embodiment the inhibition of cancer cell growth includes screening for substances which are useful for treating cancer.

A more complete appreciation of the invention and many of the attendant advantages thereof will be readily obtained as the same becomes better understood by reference to the following detailed description when considered in connection with the accompanying drawings.

### BRIEF DESCRIPTION OF THE DRAWINGS

FIG. 1. A, phase contrast microscopy (x225) of FP_{A} and FP_{B} expressing cells after treatment with either vehicle (panels a and c) or 1 μM PGF_{2α} (panels b and d) for 1 hour at 37° C. B, β-catenin FITC immunofluorescence (green) and nuclear DAPI fluorescence (blue) microscopy (x225) of FP_{A} and FP_{B} cells after the same treatment. Cells were labeled and prepared for microscopy as described in Experimental Procedures. The results are representative of more than three experiments.

FIG. 2. A, Immunoblot of β-catenin in particulate and cytosolic fractions prepared from FP_{A} and FP_{B} expressing cells after treatment with either vehicle (lanes a and c) or 1 μM PGF_{2α} (lanes b and d) for 1 hour at 37° C. B, RT-PCR of β-catenin and control glyceraldehyde-3-phosphate dehydrogenase (GAPDH) mRNA from FP_{A} and FP_{B} expressing cells after the same treatment. Immunoblotting (10 μg of protein per sample) and RT-PCR were done as described in Experimental Procedures. Results for both the immunoblotting and RT-PCR are representative of three independent experiments.

FIG. 3. Immunoblot (IB) of β-catenin (β-cat) in cytosolic fractions and nuclear extracts; and immunoblot of serine/threonine phosphorylated (PS/PT) cytosolic β-catenin, from FP_{A} and FP_{B} expressing cells after treatment with either vehicle (lanes a and c) or 1 μM PGF_{2α} (lanes b and d) for 1 hour at 37° C. Cytosolic fractions were prepared as described in Experimental Procedures and samples (100 μg protein) were immunoprecipitated (IP) with antibodies to β-catenin and were first probed with antibodies to phosphoserine and phosphothreonine (upper panel); and then were stripped and reprobed with antibodies to β-catenin (middle panel). Immunoblotting of nuclear extracts (lower panel) was done with 10 μg protein per sample without prior immunoprecipitation. Results are representative of three independent experiments.

FIG. 4. A, phase contrast microscopy (x225) and B, stimulation of Tcf/Lef responsive luciferase reporter gene activity after FP_{A} and FP_{B} expressing cells were treated with either vehicle or 1 μM PGF_{2α} for 1 hour and were washed extensively drug-free media and incubated for an additional 16 hours at 37° C. in drug-free media. The transfection conditions, drug washout, and luciferase assay are provided in Experimental Procedures. Luciferase data are normalized to the vehicle treated FP_{A} cells and are the means+/−the standard errors of three independent experiments each performed in duplicate.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. Al publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein.

As used herein PGF_{2α} is understood to mean prostaglandin F_{2α}, analogues of PGF_{2α}, and substances which mimic the action of PGF_{2α} at the FP prostanoid receptor; GPCR is understood to mean G-protein coupled receptor; Tcf is understood to mean T-cell factor; Lef is understood to mean lymphoid enhancer factor; FITC is understood to mean fluorescein isothiocyanate; DAPI is understood to mean 4′,6-diamidino-2-phenylindole; RT is understood to mean reverse transcription; PCR is understood to mean polymerase chain reaction; GSK-3β is understood to mean glycogen synthase kinase-3β; and APC is understood to mean adenomatous polyposis coli.

This invention provides a method for identifying substances potentially useful for the treatment and prevention of pre-cancerous and cancerous lesions in mammals.

In performing the present method use of cells either endogenously or exogenously expressing the FP prostanoid receptors can be used. In the case where the cell does not endogenously express the FP prostanoid receptors a suitable vector carrying the gene which encodes the FP_{A} receptor can be introduced into the cell by procedure known in the art. The vector should be suitably constructed so as to facilitate expression of the FP prostanoid receptor gene upon introduction. The gene may be maintained episomally or may be integrated into the cellular chromosomes using methods known in the art. The FP prostanoid receptor gene which can be used in accordance with the present methods are those which are isolated from mammalian species, particularly, mouse, rat, human, sheep, cow and the like.

The methods of screening substances can be performed in vitro or in vivo.

Types of assays which are embodied within the present invention include analyzing the morphology of the cell; wherein said morphology is selected from the group consisting of cell rounding, loss of filopodia, and formation of cell aggregates; wherein an absence of a change in cell morphology compared to a cell not contacted with PGF_{2α} indicates inhibition of the interaction; measuring apoptosis in said cell; assaying comprises measuring the transcription activity of a Tcf/Lef responsive promoter; or measuring the level of phosphorylation of β-catenin in the cell, wherein a increased level of phosphorylation compared to the β-catenin in a cell not contacted with said substance indicates the inhibition of the interaction. Additional detection methods for determining whether a substance successfully inhibits FP prostanoid receptors and PGF_{2α} embodied within the present invention include inositol phosphate stimulation, activation of Rho, stress fiber formation, and phosphorylation of P125 (see reference 6).

In one embodiment of the present method the cells are transfected with a reporter construct as are known in the art. Such reporter constructs are preferably sensitive to changes in -catenin signaling efficacy; typically by including a responsive promoter, e.g., a Tcf/Lef promoter. Levels of activity from the reporter construct can be determined by measuring changes in transcript levels, e.g, using Northern blots, dot-blots, primer extensions, RNase protections, RT-PCR and the like. Alternatively, the responsive promoter is functionally linked to a reporter gene whereby the levels of activity are measured by assaying changes in enzymatic, fluorescence or calorimetric activity of the reporter gene. Such reporter genes are known in the art and some examples include β-galactosidase, luciferase, green fluorescence protein and the like. These and other methods, genes, and vectors are described in, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein.

In one embodiment of the present invention the inhibition of the interaction between FP prostanoid receptors and PGF_{2α} also inhibits signaling mediated by the G-proteins G12 and G13.

This invention relates to a novel method for screening test substances for their ability to treat and prevent neoplasia, especially pre-cancerous lesions, safely. In particular, the present invention provides a method for identifying test substances that can be used to treat and prevent neoplasia, including precancerous lesions, with minimal side effects associated with inhibition and other non-specific interactions.

The method of this invention is useful to identify substances that can be used to treat or prevent neoplasms, and which are not characterized by the serious side effects of conventional NSAIDs.

Cancer and precancer may be thought of as diseases that involve unregulated cell growth. Cell growth involves a number of different factors. One factor is how rapidly cells proliferate, and another involves how rapidly cells die. Cells can die either by necrosis or apoptosis depending on the type of environmental stimuli. Cell differentiation is yet another factor that influences tumor growth kinetics. Resolving which of the many aspects of cell growth is affected by a test substance is important to the discovery of a relevant target for pharmaceutical therapy. Screening assays based on this selectivity can be combined with tests to determine which substances having growth inhibiting activity.

“Precancerous lesion” includes syndromes represented by abnormal neoplastic, including dysplastic, changes of tissue. Examples include dysplastic growths in colonic, breast, prostate or lung tissues, or conditions such as dysplastic nevus syndrome, a precursor to malignant melanoma of the skin. Examples also include, in addition to dysplastic nevus syndromes, polyposis syndromes, colonic polyps, precancerous lesions of the cervix (i.e., cervical dysplasia), esophagus, lung, prostatic dysplasia, prostatic intraneoplasia, breast and/or skin and related conditions (e.g., actinic keraosis), whether the lesions are clinically identifiable or not.

“Carcinoma” or “cancer” refers to lesions which are cancerous. Examples include malignant melanomas, breast cancer, prostate cancer and colon cancer. As used herein, the terms “neoplasia” and “neoplasms” refer to both cancerous and pre-cancerous lesions.

In an alternate embodiment, the screening method of the present invention involves further determining whether the substance reduces the growth of tumor cells. Various cell lines can be used in the sample depending on the tissue to be tested. For example, these cell lines include: colonic adenocarcinoma; lung adenocarcinoma carcinoma; breast adenocarcinoma; melanoma line; keratinocytes; prostrate carcinoma and other cancer model cell lines commonly used in the art. Cytotoxicity data obtained using these cell lines are indicative of an inhibitory effect on neoplastic lesions. These and other cell lines are well characterized, and are used commonly used in the art for screening for new anti-cancer drugs.

One embodiment of the present method of screening for substances which is useful for selecting substances for the treatment of cancer include the tumor progression model. This model includes the induction of cells into a cancerous state by applying TPA. The subsequent or concurrent administration of the tested substance and reduction in tumor progression would be indicative of the successful inhibition of the interaction between the FP prostanoid receptor and PGF_{2α}.

Significant tumor cell growth inhibition greater than about 50% at a dose of 100 μM or below is further indicative that the substance is useful for treating neoplastic lesions. Preferably, an IC₅₀ value is determined and used for comparative purposes. This value is equivalent to the concentration of drug needed to inhibit tumor cell growth by 50% relative to the control. Preferably, the IC₅₀ value should be less than 100 μM for the substance to be considered further for potential use for treating neoplastic lesions.

One measure of successful inhibition is to assay the presence or absence of apoptosis in the cell carrying the FP prostanoid receptor. Methods of detecting apoptosis include the TUNEL assay and ELISA assay. These and other methods are disclosed in Tomei, L. D. and Cope, F. O. Apoptosis: The Molecular Basis of Cell Death (1991) Cold Spring Harbor Press, N.Y.; Tomei, L. D. and Cope, F. O. Apoptosis II: The Molecular Basis of Apoptosis in Disease (1994) Cold Spring Harbor Press, N.Y.; Duvall and Wyllie (1986) Immun. Today 7(4):115-119 and Sambrook et al., Molecular Cloning: A Laboratory Manual, Cold Spring Harbor Laboratory, New York (1989).

Examples of useful substances capable of inhibiting the interaction between FP prostanoid receptors and PGF_{2α} are antibodies that bind to either FP prostanoid receptors or PGF_{2α}. In a preferred embodiment antibodies binding to the FP prostanoid receptors bind to a extracellular potion of the receptor. Such antibodies are readily obtainable by one of skill in the art using conventional antibody isolation and production methods. Such methods are described in Harlow and Lane, Antibodies: A Laboratory Manual, Cold Spring Harbor Laboratory Press, 1988, which is hereby incorporated by reference.

Upon successful isolation of a substance which can inhibit the interaction between FP prostanoid receptors and PGF_{2α} it will be useful to formulate this substance into a pharmaceutical composition suitable for administration to a animal, preferably a human. Such pharmaceutical compositions typically include pharmaceutically acceptable carriers. The pharmaceutically acceptable carrier which can be used in the present invention is not limited particularly and includes an excipient, a binder, a lubricant, a colorant, a disintegrant, a buffer, an isotonic agent, a preservative, an anesthetic, and the like which can be used in a medical field.

The pharmaceutical composition can be applied by any suitable administration method depending on the purpose of treatment and selected from injection (subcutaneous, intracutaneous, intravenous, intraperitoneal, etc.), eye dropping, instillation, percutaneous administration, oral administration, inhalation, and the like.

The dosage form such as injectable preparations (solutions, suspensions, emulsions, solids to be dissolved when used, etc.), tablets, capsules, granules, powders, liquids, liposome inclusions, ointments, gels, external powders, sprays, inhalating powders, eye drops, eye ointments, suppositories, pessaries, and the like can be selected appropriately depending on the administration method, and the inhibiting substance of the present invention can be accordingly formulated. Formulation in general is described in Chapter 25.2 of Comprehensive Medicinal Chemistry, Volume 5, Editor Hansch et al, Pergamon Press 1990.

The dose of the medicine of the present invention should be set up individually depending on the purpose of administration (prevention, maintenance (prevention of aggravation), alleviation (improvement of symptom) or cure); the kind of disease; the symptom, sexuality and age of patient; the administration method and the like and is not limited particularly.

Having generally described this invention, a further understanding can be obtained by reference to certain specific examples which are provided herein for purposes of illustration only, and are not intended to be limiting unless otherwise specified.

### EXAMPLES

Experimental Procedures

Immunofluorescence Microscopy. HEK-293 cells stably expressing the ovine FP_{A} and FP_{B} prostanoid receptor isoforms (5) were split and grown in six-well plates containing 22-mm round glass cover slips for 3-4 days. Cells were treated with either vehicle (sodium carbonate, 0.002% final) or 1 μM PGF_{2α} and were rapidly washed, fixed, and incubated with a 1:1000 dilution of a mouse monoclonal antibody to β-catenin (Transduction Laboratories). They were then washed and incubated with a 1:4000 dilution of an FITC-conjugated goat anti-mouse secondary antibody (Sigma). Nuclei were stained with 4′,6-diamidino-2-phenylindole (DAPI, Sigma). Cells were visualized by phase-contrast and epifluorescence microscopy as previously described (6).

Immunoprecipitaaion and Blotting. Cells were scraped and sonicated in a lysis buffer consisting of 20 mM Tris-HCl (pH 7.5), 10 mM EDTA, 2 mM EGTA, 2 mM phenylmethylsulfonylfluoride, 0.1 mg/ml leupeptin and 2 mM sodium vanadate. Samples were centrifuged (16,000×g) for 15 minutes at 4° C. and the supernatant (cytosolic fraction) was removed and the pellet (particulate fraction) was solubilized with lysis buffer containing 0.2% Triton X-100 and then centrifuged again to remove insoluble debris. For immunoprecipitation, samples were rotated for 2 hours at 4° C. with antibodies to β-catenin, followed by addition of protein-G sepharose (Amersham) and rotation for another hour. The sepharose was washed with lysis buffer and then resuspended with SDS-PAGE sample buffer and boiled. Samples were electrophoresed on 7.5% SDS-polyacrylamide gels, transferred to nitrocellulose membranes, and incubated with either antibodies to P-catenin or with a mixture of mouse monoclonal antibodies to phosphoserine (Sigma) and phosphothreonine (Sigma). The membranes were washed and incubated with horseradish peroxidase-conjugated goat anti-mouse secondary antibodies and were visualized by enhanced chemiluminiescence (SuperSignal, Pierce). Nuclear extracts were prepared according to the method of Dignam as modified by Westin et al. (7).

RT-PCR. RT was done using the Superscript Preamplification System (Life Technologies) and 1 μg of RNA/sample that had been pretreated with DNase I. This was followed by PCR using an initial incubation at 94° C. for 5 minutes, followed by 20 cycles of 94° C., 60° C., and 68° C. each for 2 minutes, and a final incubation at 68° C. for 10 minutes. The human β-catenin and GAPDH primner pairs were exactly according to Rezvani & Liew (8). Product sizes were 521 bp for β-catenin and 737 bp for GAPDH and were resolved by electrophoresis on 1.5% agarose gels. Preliminary experiments were done to find the optimal conditions for quantitative amplification of β-catenin and GAPDH rnRNA.

Tcf/Lef Reporter Gene Assay. Cells were split into 10-cm dishes and the next day were transiently transfected using FuGENE-6 (Roche) and either 10 jLg/dish of the wildtype Tcf/Lef reporter plasmid, TOPflash, or the mutant plasmid, FOPflash. FOPflash differs from TOPflash by mutation of its Tcf binding sites and serves to differentiate Tcf/β-catenin mediated signaling from background (Upstate Biotechnology). Cells were incubated overnight and were treated for 1 hour at 37° C. with either vehicle or 1 JμM PGF_{2α}. They were then rapidly washed three times each with 2 ml of Opti-MEM (Life Technologies) as previously described (6) and incubated for 16 hours at 37° C. in 10 ml of Opti-MEM containing 250 μg/ml geneticin, 200 μg/ml hygromycin B, and 100 μg/ml gentamicin. Cells were placed on ice, rinsed twice with ice cold PBS, and extracts were prepared using the Luciferase Assay System (Promega). Luciferase activity in the extracts (˜500 ng protein/sample) was measured using a Turner TD-20/20 luminometer and was corrected for background by subtraction of FOP-FLASH values from corresponding TOP-FLASH values.

Results

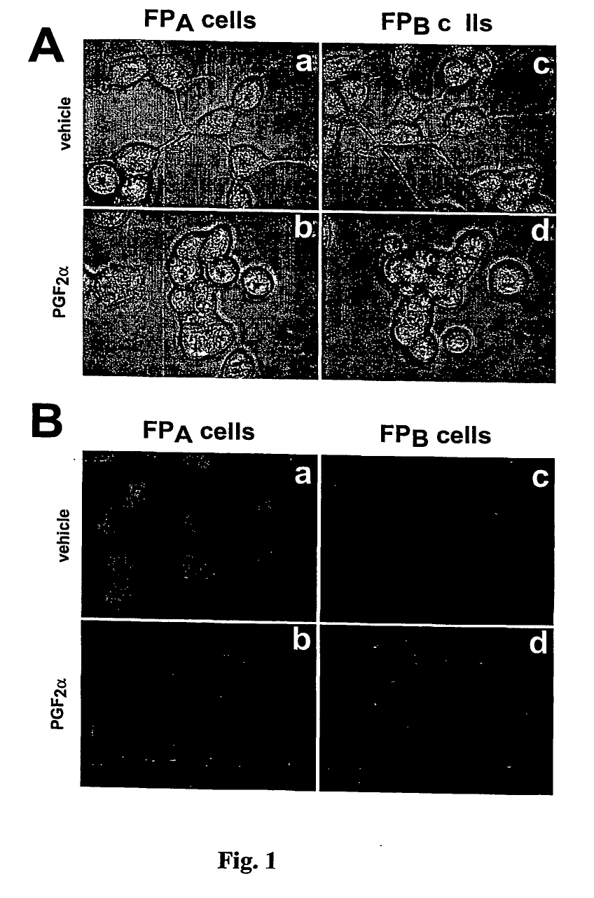

FIG. 1A shows phase contrast microscopy of HEK cells stably expressing either the ovine FP_{A} prostanoid receptor (panels a and b) or the ovine FP_{B} prostanoid receptor (panels c and d) following 1 hour treatment with either vehicle (panels a and c) or 1 μM PGF_{2α} (panels b and d). It can be appreciated that in both FP_{A} and FP₁ expressing cells treatment with PGF_{2α} resulted in morphological changes consisting of a loss of filopodia and formation of cell aggregates. We have previously shown that these morphological changes involve the activation of Rho and phosphorylation of p125 focal adhesion icinase (5). However, following the removal of PGF_{2α} the FPA expressing cells show a rapid (within 1 hour) reversal of these morphological changes, whereas the FP_{B} expressing cells remain rounded even after 48 hours (6). To investigate the possible role of other adhesion proteins in this process we used immunofluorescence microscopy to examine the localization of E-cadherin and β-catenin in HEK cells stably expressing either the FP_{A} or FP_{B} isoforms following treatment with 1 μM PGF_{2α}. Although effects on E-cadherin localization were not apparent (data not shown), FIG. 1B shows that PGF_{2α} treatment resulted in a marked accumulation of β-catenin in regions of cell-to-cell contact in FP_{B} expressing cells (panels c and d), but not in FP_{A} expressing cells (panels a and b). Both cells lines, however, showed agonist dependent cell rounding following treatment with PGF_{2α}. (FIG. 1A) indicating that the process of cell rounding itself was not responsible for the increased contiguous accumulation of β-catenin in the FP_{B} expressing cells.

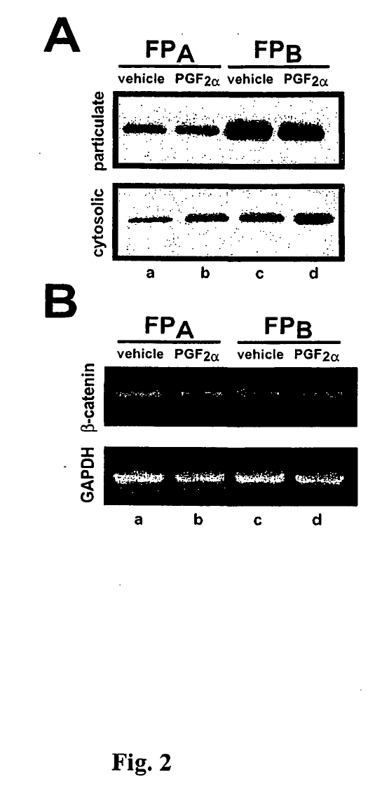

Besides its role in cell adhesion β-catenin is well recognized as a signaling molecule that undergoes stimulus dependent translocation from the cytosol to the nucleus where it is involved in the regulation of Tcf/Lef mediated gene transcription (9-11). We, therefore, used immunoblotting to examine both particulate and cytosolic fractions for changes in β-catenin expression following treatment of FP_{A} and FP_{B} expressing cells with PGF_{2α}. FIG. 2A shows that the expression of β-catenin is higher in both the particulate and cytosolic fractions from FP_{B} expressing cells as compared with FP_{A} expressing cells. Furthermore, treatment with PGF_{2α} increased the levels of cytosolic β-catenin in both the FP_{A} and FP_{B} expressing cells, but had little effect on the levels of β-catenin in the particulate fraction. Reverse transcription OM) followed by the polymerase chain reaction (PCR) was used to determine if there were any differences in β-catenin MnRNA levels under these same experimental conditions. FIG. 2B shows that β-catenin and GAPDH MRNA levels were the same for both cell lines and were not affected by PGF_{2α}, indicating that the observed differences in β-catenin expression appear to be the result of changes in translation and/or protein turnover.

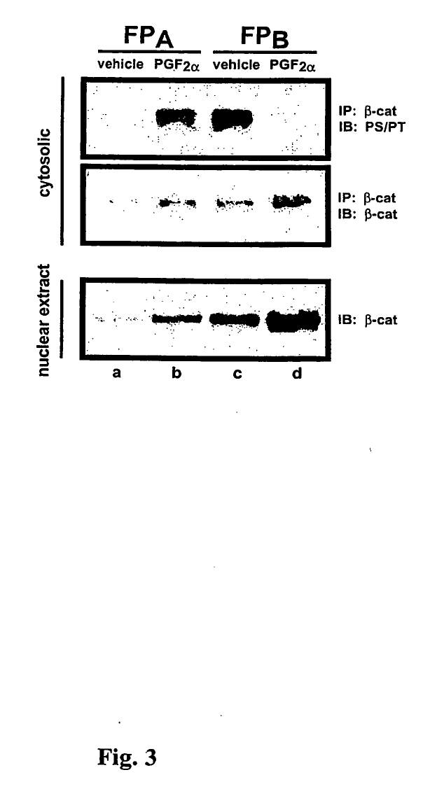

Serine/threonine phosphorylation of β-catenin by glycogen synthase kinase-3β (GSK-3β) marks β-catenin for degradation and is a critical factor in the regulation of its signaling activity (12,13). Thus, under most conditions cytosolic β-catenin is phosphorylated leading to an association with the tumor suppressor protein, adenomatous polyposis coli (APC), and the scaffolding protein, axin, which is then followed by ubiquitination and proteasomal degradation (14). Using immunoprecipitation and immunoblotting we examined serine/threonine phosphorylation of β-catenin following treatment of either FP_{A} or FP_{B} expressing cells with PGF_{2α}. FIG. 3 shows that in FP_{A} expressing cells the vehicle control levels of cytosolic β-catenin are very low and there is no detectable phosphorylation (lane a). Following treatment with PGF_{2α} the levels of cytosolic β-catenin increase and there is a marked increase in phosphorylation (lane b). In FP_{B} expressing cells the vehicle control levels of cytosolic β-catenin are already elevated and so is phosphorylation (lane c). This probably reflects endogenous GSK-3β activity and tight coupling to the elevated levels of cytoplasmic β-catenin. After treatment of FP_{B} expressing cells with PGF_{2α}, however, there is a further increase in cytosolic β-catenin, but a dramatic fall in phosphorylation (lane d), suggestive of an uncoupling or decrease in GSK-3β activity. It would, therefore, be expected that degradation of cytosolic β-catenin would be favored at the expense of nuclear translocation in FP_{A} expressing cells, whereas, the opposite would be true in FP_{B} expressing cells. This appears to be confirmed in FIG. 3 where immunoblotting of nuclear extracts shows significantly higher levels of β-catenin in FP_{B} expressing cells following treatment with PGF_{2α} (lane d) as compared with FP_{A} expressing cells (lane b).

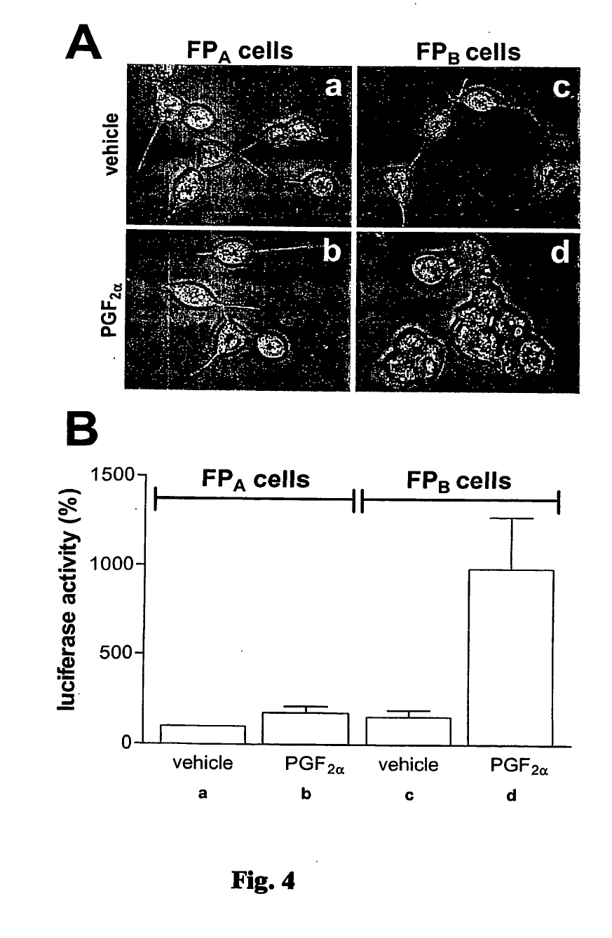

Following nuclear translocation, β-catenin is known to interact with members of the Tcf/Lef family of transcription factors (15), which in many instances serves as a switch for cellular differentiation and transformation. Because of this signaling potential, we were interested in the possibility that the failure of FP_{B} expressing cells to return to their original dendritic morphology following removal of PGF, might represent a transformation event induced by a β-catenin mediated switch in gene expression. To examine this we transiently transfected either FP_{A} or FP_{B} expressing cells with a Tcf/Lef responsive reporter plasmid (16) and measured luciferase reporter gene activity following treatment with 1 μM PGF_{2α}. Initially we found that basal levels of luciferase activity were elevated (˜3 fold) in FP_{B} expressing cells as compared with FP_{A} expressing cells and that measurement of reporter gene activity immediately following a 1 hour treatment with PGF_{2α} did not stimulate luciferase activity in either cell line (data not shown). However, as shown in FIG. 4A, the morphological effects of PGF_{2α} on FP_{B} expressing cells persist long after its removal. Thus, when cells are examined 16 hours after an initial 1 hour treatment with PGF_{2α} (followed by washout and replacement with fresh media), the FP_{A} expressing cells show a return to their original dendritic morphology (panel b), whereas, the FP_{B} expressing cells remain rounded and aggregated (panel d). We, therefore, examined Tcf/Lef reporter gene activity at the same time point and the results are shown in FIG. 4B. In a remarkable parallel to the morphological findings, FP_{B} expressing cells show a persistent activation of luciferase activity (column d) that is roughly 6.5 fold higher than either the vehicle control (column c) or PGF_{2α} treated FP_{A} cells (column b). We have previously reported that the failure of FP_{B} expressing cells to show reversal of cell rounding is not because of changes in the kinetics of PGF_{2α} binding or in its removal during the washout procedure (6).

Discussion

The present inventor has shown that FP_{B} expressing cells differ in several important regards from FP_{A} expressing cells in terms of their potential for activation of Tcf/β-catenin mediated signaling. First FP_{B} expressing cells show PGF_{2α} stimulated accumulation of β-catenin at their contiguous cell boundaries that is not evident in FP_{A} expressing cells. Second, while both FP_{A} and FP_{B} expressing cells show PGF_{2α} stimulated increases in cytosolic β-catenin, in FP_{A} expressing cells this is accompanied by increased β-catenin phosphorylation and in FP_{B} expressing cells by decreased β-catenin phosphorylation. Third, FP_{B} expressing cells show a profound stimulation of Tcf/Lef reporter gene activity 16 hours after agonist removal that is essentially absent in FP_{A} expressing cells. Obviously a key control point could be in the differential phosphorylation of β-catenin. Thus, it is possible that the agonist stimulated accumulation of β-catenin at the contiguous cell boundaries of FP_{B} cells results in enhanced adhesive interactions with E-cadherin. In turn, this could initiate E-cadherin outside-in signaling leading to the sequential activation of phosphatidylinositol 3-kinase and Akt kinase (17). This is potentially meaningful because phosphorylation of GSK-3β by Akt kinase is inhibitory (18) and could lead to the decreased phosphorylation of β-catenin found in agonist treated FP_{B} cells.

Recently Meigs et al. reported that constitutively active mutants of G_{α12} and G_{α13} interact with the cytoplasmic domain of ε-cadherin resulting a release of β-catenin and stimulation of Tcf/Lef reporter gene activity in a mutant cell line lacking APC (19). This is an intriguing finding since it is the first report of a link between heterotrimeric G-proteins and the Tcf/β-catenin signaling pathway. However, because of the altered nature of their model, its physiological relevance might be questioned. In light of the present findings, though, it appears likely that both GPCRs and heterotrimeric G-proteins will be involved with activation of this important signaling pathway. We previously showed that FP receptors activate Rho and hypothesized that this occurred through activation of G₁₂ and/or G₁₃ (5). Both receptor isoforms were equally effective in this regard and, therefore, it would appear unlikely that activation of G₁₂ and/or G₃ could be solely responsible for the present findings since persistent activation of Tcf/β-catenin signaling was only observed for cells expressing the FP_{B} isoform.

One possible mechanism for that activation of Tcf/β-catenin signaling by PGF_{2α} in cells expressing the FP_{B} receptor is responsible for a phenotypic transformation that is morphologically similar, but fundamentally different from the cell rounding observed in agonist treated FP_{A} cells. Thus, maintenance of shape change in FP_{A} expressing cells depends upon continuous stimulation by PGF_{2α} and following its removal the cells revert back to their original morphology. In contrast, while shape change in FP_{B} expressing cells is initiated by PGF_{2α} its maintenance is independent of further PGF_{2α} stimulation and may not even require the continued presence of the receptor following the initial agonist stimulation. In this manner the FP_{B} prostanoid receptor is functioning as one would expect of a trigger in a developmental or malignant transformation pathway.

This has considerable significance for the signaling potential of FP prostanoid receptors and possibly for other GPCRS as well. For example, in sheep and cattle it is known that PGF_{2α} is the physiological signal for regression of the corpus luteum, but only during a short window of the luteal cycle. Thus, if pregnancy occurs the corpus luteum is maintained and loses sensitivity to the luteolytic actions of PGF_{2α} (20). Interestingly the expression of FP receptors does not appear to change during this transition, however, these receptors are represented almost entirely by the FP_{A} isoform (21). Brief expression of a small population of FP_{B} receptors during the sensitive phase luteal cycle could explain the luteolytic actions of PGF_{2α}. Indeed the FP_{B} isoform was cloned from a midphase ovine corpus luteum cDNA library where the predominant isoform was the FP_{A} (1).

Another condition that might involve the FP_{B} isoform or a homologue is in colorectal cancer. It is well established that aberrant activation of Tcf/β-catenin signaling is strongly associated with the development of this disease (22-24) and that inhibition of cyclooxygenase by nonsteroidal antiinflammatory drugs (NSAIDs) can slow tumor progression (25). However, the specific mechanism of this beneficial effect is vague because of the large number of prostanoid metabolites that are affected by the inhibition of cyclooxygenase. The disclosure provided in the present application support a mechanism in which NSAID mediated decreases in PGF_{2α}, would result in decreased Tcf/β-catenin signaling by FP_{B} prostanoid receptors. This conclusion is supported by animal models of skin carcinogenesis in which PGF₂, reversed the anti-tumor promoting activity of indomethacin and was the only prostanoid tested that increased the tumor promoting activity of phorbol esters (26). Although a human homologue of the ovine FP_{B} receptor has not yet been identified, it is easy to imagine genetic or even posttranslational mechanisms that could give rise to functional FP_{B} isoforms. Thus, much like the known mutations of APC, truncation of the human FP_{A} receptor by allelic variation, somatic mutations, or proteolytic cleavage could give rise to receptors capable of producing persistent activation of Tcf/β-catenin signaling. The possible role of FP_{B} receptors in these and other physiological processes is intriguing and awaits future studies.

Another condition that might involve the FP_{B} isoform or a homologue is in the control of hair growth. Thus, it has been well documented that in some patients receiving latanoprost (an analogue of PGF_{2α}), for the treatment of glaucoma, there is hypertrichosis of the eyelashes and adjacent hair in the treated eye, but not in the untreated eye (27-28). The mechanism of this curious side effect is unknown; however, recent studies have indicated that Wnt signaling and activation of Tcf/Lef transcription complexes is critical to hair follicle development and differentiation (29-30). The disclosure provided in the present application supports a mechanism in which activation of the FP_{B} receptor or a homologue would stimulate a Tcf/β-catenin signaling pathway in the hair follicle leading to increased hair growth.

Obviously, numerous modifications and variations on the present invention are possible in light of the above teachings. It is therefore to be understood that within the scope of the appended claims, the invention may be practiced otherwise than as specifically described herein.

### REFERENCES

1. Pierce, K. L., Bailey, T. J., Hoyer, P. B., Gil, D. W., Woodward, D. F., and Regan, J. W. (1997) J. Biol. Chem. 272, 883-887

2. Namba, T., Sugimoto, Y., Negishi, M., Irie, A., Ushikubi, F., Kakizuka, A., Ito, S., Ichikawa, A., and Narumiya, S. (1993) Nature 365, 166-170

3. Raychowdhury, M. K., Yukawa, M., Collins, L. J., McGrail, S. H., Kent, K. C., and Ware, J. A. (1994) J. Biol. Chem. 269, 19256-19261

4. Pierce, K. L., and Regan, J. W. (1998) Life Sci. 62, 1479-1483

5. Pierce, K. L., Fujino, H., Srinivasan, D., and Regan, J. W. (1999) J. Biol. Chem. 274, 35944-35949

6. Fujino, H., Pierce, K. L., Srinivasan, D., Protztnan, C. E., Krauss, A. H., Woodward, D. F., and Regan, J. W. (2000) J. Biol. Chem. 275, 29907-29914

7. Westin, G., Gester, T., Muller, M. M., Schaffner, G., and Schaffner, W. (1987) Nucleic Acids Res. 15, 6787-6798

8. Rezvani, M., and Liew, C. C. (2000) J. Biol. Chem. 275, 18470-18475

9. Cadigan, K. M., and Nusse, R. (1997) Genes Dev. 11, 3286-3305

10. Dale, T. C. (1998) Biochem J. 329, 209-223

11. Barker, N., Morin, P. J., and Clevers, H. (2000) Adv Cancer Res. 77, 1-24

12. Rubinfeld, B., Albert, I., Porfiri, E., Fiol, C., Munenaitsu, S., and Polakis, P. (1996) Science 272, 1023-1026

13. Aberle, H., Bauer, A., Stappert, J., Kispert, A., and Kemler, R. (1997) EMBO J 16, 379714.

14. Ikeda, S., Kishida, S., Yamamoto, H., Murai, H., Koyama, S., and Kikuchi, A. (1998) EMBO J 17, 1371-1384

15. Behrens, J., von Kries, J. P., Kuhl, M., Bruhn, L., Wedlich, D., Grosschedl, R., and Birchmeier, W. (1996) Nature 382, 638-642

16. Van de Wetering, M., Cavallo, R., Dooijes, D., van Beest, W., van Es, J., Loureiro, J., Ypma, A., Hursh, D., Jones, T., Bejsovec, A., Peifer, M., Mortin, M., and Clevers, H. (1997) Cell 88, 789-799

17. Pece, S., Chiariello, M., Murga, C., and Gutkind, S. (1999) J. Biol. Chem. 274, 19347-19351

18. Cross, D. A., Alessi, D. R., Cohen, P., Andjelkovich, M., and Hemmings, B. A. (1995) Nature 378, 785-789

19. Meigs, T. E., Fields, T. A., McKee, D. D., and Casey, P. J. (2001) Proc. Natl. Acad Sci. USA 98, 519-524

20. Silvia, W. J., and Niswender, G. D. (1986) J Anim Sci 63, 1201-1207

21. Rueda, B. R., Botros, I. W., Pierce, K. L., Regan, J. W., and Hoyer, P. B. (1995) Endocrine 3, 781-787

22. Korinek, V., Barker, N., Morin, P. J., van Wichen, D., de Weger, R., Kinzler, K. W., Vogelstein, B., and Clevers, H. (1997) Science 275, 1784-1787

23. Morin, P. J., Sparks, A. B., Korinek, V., Barker, N., Clevers, H., Vogelstein, B., and Kinzler, K. W. (1997) Science 275, 1787-1790

24. Peifer, M., and Polakis, P. (2000) Science 287, 1606-1609

25. Smalley, W. E., and Dubois, R. N. (1997) Adv. Pharmacol. 39, 1-20

26. Furstenberger, G., Gross, M., and Marks, F. (1989) Carcinogenesis 10, 91-96

27. Mansberger, S. L., and Cioffi, G. A. (2000) Arch. Ophthal. 118, 718-719

28. Johnstone, M. A. (1997) Am. J Ophthal 124:544-547

29. DasGupta, R., and Fuchs, E. (1999) Development 126, 45574568

30. Kulessa, H., Turk, G., and Hogan, F. L. M. (2000) EMBO J 19, 6664-6674

1. A method of screening for substances which inhibit the interaction between a FP prostanoid receptors and a compound having PGF_{2α} activity comprising
contacting a cell expressing the FP prostanoid receptors with the substance to be screened;
contacting said cell with said PGF_{2α} compound; and
assaying the presence or absence of interaction between FP prostanoid receptors and PGF_{2α} compound, wherein the absence of an interaction between FP prostanoid receptors and PGF_{2α} compound indicates the substance inhibits the interaction.

2. The method of claim 1, wherein said assaying comprises analyzing the morphology of the cell; wherein said morphology is selected from the group consisting of cell rounding, loss of filopodia, and formation of cell aggregates; wherein an absence of a change in cell morphology compared to a cell not contacted with PGF_{2α} compound indicates inhibition of the interaction.

3. The method of claim 1, wherein the assaying comprises measuring apoptosis in said cell.

4. The method of claim 1, wherein said cell endogenously expresses the FP prostanoid receptor.

5. The method of claim 1, wherein said assaying comprises measuring the transcription activity of a Tcf/Lef responsive promoter.

6. The method of claim 5, wherein said assaying comprises a detection method selected from the group consisting of RT-PCR, Northern blot, luciferase reporter gene, β-gal reporter gene, and other reporters.

7. The method of claim 1, wherein said assaying comprises measuring the level of phosphorylation of β-catenin in the cell, wherein an increased level of phosphorylation compared to the β-catenin in a cell not contacted with said substance indicates the inhibition of the interaction.

8. The method of claim 1, wherein said substance is an antibody.

9. The method of claim 8, wherein said antibody binds to the FP prostanoid receptor.

10. The method of claim 8, wherein said antibody binds to PGF_{2α} compound.

11. The method of claim 1, wherein said PGF_{2α} compound is PGF_{2α}.

12. The method of claim 1, wherein said assaying comprises measuring changes in at least one member selected from the group consisting of inositol phosphate stimulation, activation of Rho, stress fiber formation and phosphorylation of P125.

13. The method of claim 1, wherein said FP prostanoid receptor is FP_{B}.

14. A method of screening for substances which inhibit the interaction between a FP prostanoid receptor and a compound having PGF_{2α} activity comprising
introducing and expressing a polynucleotide which encodes the FP prostanoid receptor;
contacting said cell expressing the FP prostanoid receptor with the substance to be screened;
contacting said cell with said PGF_{2α} compound; and
assaying the presence or absence of interaction between FP prostanoid receptor and PGF_{2α} compound, wherein the absence of an interaction between FP prostanoid receptor and PGF_{2α} indicates the substance inhibits the interaction.

15. A method of inhibiting the interaction between FP prostanoid receptor and a compound having PGF_{2α} activity comprising contacting said FP_{A} with a substance which is capable of inhibiting said interaction.

16. A method of inhibiting β-catenin signaling comprising contacting a cell expressing FP prostanoid receptor with a substance which is capable of inhibiting the interaction between FP prostanoid receptor and a compound having PGF_{2α} activity.

17. A method of inhibiting G12 and G13 mediated signaling comprising contacting a cell expressing FP prostanoid receptor with a substance which is capable of inhibiting the interaction between FP prostanoid receptor and a compound having PGF_{2α} activity.

18. A method of treating cancer comprising administering to a patient in need thereof a substance which inhibits the interaction between FP prostanoid receptor and a compound having PGF_{2α} activity in an amount sufficient to inhibit said interaction.

19. The method of claim 18, wherein said cancer is colorectal cancer.

20. A method of screening for substances which inhibit β-catenin signaling comprising
contacting a cell expressing FP prostanoid receptor with the substance to be screened;
contacting said cell with a compound having PGF_{2α} activity; and
assaying the signaling activity, phosphorylation and/or the subcellular localization of the β-catenin; wherein a change in one or more of the signaling activity, phosphorylation and/or the subcellular localization is lower than the signaling activity, phosphorylation and/or the subcellular localization compared to a cell not contacted with the substance indicates the substance inhibits the interaction β-catenin signaling.

21. A method of screening for a substance for their ability to inhibit cancer cell growth comprising
contacting a cell expressing FP prostanoid receptor with the substance to be screened;
contacting said cell with a compound having PGF_{2α} activity; and
assaying the change in cell growth, wherein a decrease in cell growth is indicates an inhibition of cancer cell growth.

22. The method of claim 21, wherein said assaying comprises analyzing the morphology of the cell; wherein said morphology is selected from the group consisting of cell rounding, loss of filopodia, and formation of cell aggregates; wherein an absence of a change in cell morphology compared to a cell not contacted with said PGF_{2α} compound indicates inhibition of the interaction.

22. The method of claim 21, wherein the assaying comprises measuring apoptosis in said cell.

23. The method of claim 21, wherein said cell endogenously expresses the FP prostanoid receptor.

24. The method of claim 21, wherein said assaying comprises measuring the transcription activity of a Tcf/Lef responsive promoter.

25. The method of claim 24, wherein said assaying comprises a detection method selected from the group consisting of RT-PCR, Northern blot, luciferase reporter gene, β-gal reporter gene, and other reporters.

26. The method of claim 21, wherein said assaying comprises measuring the level of phosphorylation of β-catenin in the cell, wherein an increased level of phosphorylation compared to the β-catenin in a cell not contacted with said substance indicates the inhibition of the interaction.

27. The method of claim 21, wherein said substance is an antibody.

28. The method of claim 27, wherein said antibody binds to the FP prostanoid receptor.

29. The method of claim 27, wherein said antibody binds to PGF_{2α} compound.

30. The method of claim 21, wherein said PGF_{2α} compound is PGF_{2α}.

31. The method of claim 21, wherein said assaying comprises measuring changes in at least one member selected from the group consisting of inositol phosphate stimulation, activation of Rho, stress fiber formation and phosphorylation of P125.

32. The method of claim 21, wherein said FP prostanoid receptor is FP_{B}.

33. The method of claim 21, wherein said inhibiting cancer cell growth comprises treating cancer.

34. The method of claim 21, wherein said method is performed in vitro or in vivo.
